(19) United States
(12) Patent Application Publication
(10) Pub. No.: US 2025/0325172 A1
(43) Pub. Date: Oct. 23, 2025

(54) FILTER CHANGING APPARATUS FOR AN ENDOSCOPIC CAMERA, CAMERA HEAD FOR AN ENDOSCOPE, AND RETROFIT KIT FOR RETROFITTING A CAMERA HEAD AND/OR AN ENDOSCOPE

(71) Applicant: KARL STORZ SE & Co. KG, Tuttlingen (DE)

(72) Inventors: Max LEMPERLE, Tuttlingen (DE); Florian HUBER, Tuttlingen (DE); Jonas DAVID, Tuttlingen (DE)

(21) Appl. No.: 19/182,717

(22) Filed: Apr. 18, 2025

(30) Foreign Application Priority Data
Apr. 18, 2024 (DE) 10 2024 110 878.9

(51) Int. Cl. A61B 1/00; A61B 1/04

(52) U.S. Cl. CPC A61B 1/00186; A61B 1/00105; A61B 1/042

(57) ABSTRACT

The invention relates to a filter changing apparatus for an endoscopic camera, wherein the filter changing apparatus has a housing having at least one first housing part, an optical passage along an optical axis, a rotatable drive gear, and at least two filter holder arms each having a filter receptacle for an optical filter having a filter center axis, wherein the at least two filter holder arms are each rotatably arranged on the first housing part by means of a rotary shaft having an axis of rotation, wherein the respective axis of rotation is spaced apart from the filter center axis of each filter holder arm and the axes of rotation are arranged in parallel with one another, wherein, on each filter holder arm, a filter gear is arranged around the respective rotary shaft, and the rotatable drive gear can engage in each filter gear, so that, when the rotatable drive gear is driven solely by engaging with the filter gears, the at least two filter holder arms can be simultaneously rotated and/or pivoted into and/or out of the optical passage. Furthermore, the invention relates to a camera head and to a retrofit kit for retrofitting a camera head and/or an endoscope.

### CROSS-REFERENCE TO RELATED APPLICATIONS

This Application claims the benefit Under 35 U.S.C. 119(a) to German Patent Application No. 10 2024 110 878.9, filed Apr. 18, 2024, the disclosure of which is incorporated herein by reference in its entirety.

### SUMMARY

The invention relates to a filter changing apparatus for an endoscopic camera, wherein the filter changing apparatus has a housing having at least one first housing part, an optical passage along an optical axis, a rotatable drive gear, and at least two filter holder arms each having a filter receptacle for an optical filter having a filter center axis, wherein the at least two filter holder arms are each rotatably arranged on the first housing part by means of a rotary shaft having an axis of rotation, wherein the respective axis of rotation is spaced apart from the filter center axis of each filter holder arm, and the axes of rotation are arranged in parallel with one another. Furthermore, the invention relates to a camera head for an endoscope and to a retrofit kit for retrofitting a camera head and/or an endoscope.

In medical and non-medical applications, observation instruments such as endoscopes are used to examine internal cavities of a human or animal body or of an industrial, technical item, such as a pipeline. For imaging, a camera head having an image sensor can be used together with the endoscope. In order to improve the image quality and/or make different observation modes possible, it is known to introduce different filters into the beam path of the observation instrument.

For example, in fluorescence imaging, the item to be examined is exposed to light with an excitation radiation, which excites a fluorophore, which has been previously applied to the item or is already present, to emit light of a certain emission wavelength, wherein the excitation wavelength and the emission wavelength are usually different. Normally, the emission wavelength is longer than the excitation wavelength. The emitted emission light is usually significantly weaker than other light sources, such as excitation fluorescence light or imaging white light. For these reasons, it is necessary to filter out unwanted wavelength bands by means of a filter so that, if possible, only the desired spectrum and/or the emission wavelength of the fluorophore reaches the camera head in fluorescence mode. For changing between different observation modes, two or more filters are usually introduced one after the other into the beam path of the observation optics, for which purpose various filter changers are known in principle.

DE 101 57 057 A1 discloses an apparatus for positioning at least one optical component within an endoscopic system with a housing, through which the optical axis of the endoscopic system runs and in which the at least one component is arranged, which can be pivoted into the beam path and out of it again about a pivot axis running substantially in parallel with a longitudinal axis of the housing, wherein the at least one component, e.g., a filter for a specific spectral wavelength range, is arranged on a carrier pivotable about the pivot axis. In this case, a smallest distance of an inner wall of the housing from the pivot axis is smaller than a greatest distance of the pivot axis to an outer edge of the at least one component. Due to the closer arrangement of the pivot axis of each pivotable carrier to the inner wall of the housing, the number of carriers for holding as many different filters as possible is limited by the spatially extensive pivoting movement within the limited space of the housing. In addition, the housing is firmly connected to a housing of the optical head of the endoscope. Another disadvantage is that, due to the pivot axis being arranged spatially closer to the inner wall of the housing, a plurality of movable individual parts are required in order to introduce a plurality of filters into the beam path. This results in increased costs and installation effort. In addition, this increases the risk of wear, inaccuracies in the particular pivoting movement, and consequently malfunctions.

From the applicant's own prior art (German application number 10 2022 131 502.9), a filter changing apparatus for an endoscopic camera head with at least three optical filters is known, which filter changing apparatus has a groove with a non-circular guide track and a rotating element, wherein, by rotating the rotating element, carriers arranged one after the other in the groove are moved in each case for one filter or a pivoting movement of a carrier arm into and out of the beam path takes place by means of a guide element partially arranged in the groove, due to an at least partial movement along the non-circular guide track. The disadvantage here is that, in order to arrange a plurality of filters, a carrier arm having a guide element is required in each case and therefore, due to the larger number of guide elements or filter carriers arranged directly in the groove, disruptive influences on the function of the filter changing apparatus can occur due to the frictional forces that occur. The high frictional forces can cause the movement mechanism to jam and/or wear and adversely affect the inward and outward pivoting behavior and the locking position, so that a malfunction or defect in the filter changing apparatus can occur. In addition, the pivotable carrier arms overlap, so that a larger installation space is required along the optical axis.

A general disadvantage of known filter changing apparatuses with pivotable carrier arms is that either only one carrier arm is pivoted into and out of the optical input and the other carrier arms are in a rest position, or two or more pivotable carrier arms, in which one pivots in when the other pivots out, must be coordinated at the same time. This leads either to a loss of time when changing the filter due to the movement of only one carrier arm or, especially when there are more than three pivotable carrier arms, to a high degree of complexity caused by the carrier arms simultaneously moving and/or being driven individually, but in a coordinated fashion. When two or more carrier arms move simultaneously, a large installation space transverse to the optical axis and thus a large diameter of the filter changing apparatus is usually necessary to avoid a collision between the carrier arms that are moving at the same time.

The object of the invention is to improve upon the prior art.

The object is achieved by a filter changing apparatus for an endoscopic camera, wherein the filter changing apparatus has a housing having at least one first housing part, an optical passage along an optical axis, a rotatable drive gear, and at least two filter holder arms, each having a filter receptacle for an optical filter having a filter center axis, wherein the at least two filter holder arms are each rotatably arranged on the first housing part by means of a rotary shaft having an axis of rotation, wherein the respective axis of rotation is spaced apart from the filter center axis of the respective filter holder arm and the axes of rotation are parallel to one another, wherein, on each filter holder arm, a filter gear is arranged around the rotary shaft, and the rotatable drive gear is engageable with the filter gear, so that, when the rotatable drive gear is driven solely by engagement with the filter gears, the at least two filter holder arms can be simultaneously rotated and/or pivoted into and/or out of the optical passage.

Thus, a filter changing apparatus is provided with which at least two or more filters can be pivoted quickly and in direct succession into the beam path of an endoscopic camera due to the simultaneous movement of all the filter holder arms. Since the rotatable drive gear engages, as the only drive, with all the filter gears simultaneously and thus moves the filter holder arms simultaneously, the filter holder arms are simultaneously pivoted in and out quickly, precisely, and efficiently. This thus makes it possible to quickly and clearly, due to a predefined sequence with which the single drive gear engages with the filter gears, change between different filters. Above all, by simply rotating the single drive wheel clockwise and/or counterclockwise, a predefined sequence of filters and a quick change between different filters are possible, thus enabling different observation modes and imaging in quick succession.

It is particularly advantageous that the filter changing apparatus enables simultaneous movement of all the filter holder arms, but requires only a single drive gear and the desired number of filter holder arms, each having a filter gear, without the need for a plurality of complex, additional transmission components. In addition, this minimizes wear even during long operating times, thus ensuring a long service life and stable function of the filter changing apparatus.

Due to the simple drive mechanism and the specification of the movement space by the one rotary drive gear and the dimensions of the filter holder arms having the respective filter gears, the filter changing apparatus can be easily and reliably automated and adapted in terms of its size and/or to the number of filter holder arms.

By arranging the filter gears and the single drive gear in the manner of a planetary gear train, all the filter gears move continuously and sequentially along the teeth and/or the outer circumference of the drive gear, wherein each filter holder arm and thus the associated optical filter performs a complete circular movement in order to pivot in and out. Since the direction and sequence of movement of the filter holder arms are specified by the single drive gear engaging with the filter gears, the simultaneous movements of the filter holder arms can be optimally adjusted and/or coordinated by spatially arranging the rotary shafts and/or axes of rotation of the filter holder arms in relation to the drive gear, thus preventing a collision of the filter holder arms. It is particularly advantageous that the filter holder arms and thus the filters perform precisely coordinated and successive circular movements and thus do not move past one another and/or perform overlapping pivoting movements in their respective pivoting radii, which has the disadvantage of a high risk of collision. In contrast, the filter changing apparatus according to the invention in particular avoids a collision, because the filter holder arms move one after the other in continuous circular movements along the circular path of the drive gear and because their axes of rotation are always parallel to one another.

Due to the coordinated spatial arrangements and the simultaneous movements of the filter holder arms and their arrangement by means of the rotary shaft on only one housing part and thus only on the inside of one side of the housing, a very compact design of the filter changing apparatus is realized. Above all, the filter changing apparatus thus has a small dimension along the optical axis and a small diameter transverse to the optical axis.

An essential concept of the invention relates to the arrangement of at least two filter holder arms that are rotatable about their axes of rotation only on one distal-side or proximal-side housing part, and driving and moving them via their respective filter gears simultaneously and in a coordinated manner with one another by means of a single drive gear, wherein, due to the continuous engagement between the teeth of the drive gear and the teeth of all the filter gears, all the filter holder arms simultaneously move and/or pivot in and/or out and in a coordinated manner with one another. In conjunction with the arrangements of the at least two filter holder arms, each of which is infinitely rotatable, on one of the housing parts by means of a rotary shaft and the engagement of the teeth of the drive gear with the teeth of the respective filter gear of a filter holder arm, a sequence and a temporal progression of the movements of all the filter holder arms and a sequence with which the filters are changed when driving the drive gear are specified. Thus, a filter changing apparatus is provided with which different filters, in particular a plurality of fluorescence filters and/or a white light filter, can be successively pivoted into and out of the beam path of an endoscopic camera in the optical passage quickly, precisely, and efficiently. This ensures a long service life of the filter changing apparatus. In addition to the quick filter change, the frequency of use of a particular filter can also be specified by inserting the same and/or different filters into the filter receptacles of the filter holder arms. For example, an observation mode with normal white light is usually used more often than fluorescent light. Advantageously, a white light filter can thus be arranged upstream and/or downstream of a fluorescence filter in filter receptacles of successive filter holder arms, so that, in addition to the desired more frequent use of white light by respectively changing to a white light filter, the user can also clearly visually see the change between two other identical or different consecutive filters-for example, two fluorescence filters. This reduces the risk of inadvertently working in the wrong observation mode.

The following terminology is explained:

A “filter changing apparatus” (also called a “filter changer”) is in particular an apparatus by means of which at least one of two or more filters can be moved into and out of the optical beam path. By means of the filter changing apparatus, two or more filters are pivoted into and/or back out of the optical passage, in particular individually and in succession, wherein all the filter holder arms in particular move simultaneously. The filter changing apparatus can in particular be activated manually or automatically to change the filters by driving and/or rotating the rotatable drive gear. Thus, a filter can be pivoted into and out of the optical passage automatically or manually by driving and/or rotating the drive gear. This makes it possible to change between at least two filter receptacles and/or filters of two filter holder arms, whose filter gears are continuously moved by engaging with the drive gear. The filter changing apparatus in particular has at least two optical filters, preferably at least three or four and optionally more optical filters. Optionally, the filter changing apparatus can also have a filter receptacle which does not have an optical filter and thus allows free passage through the optical beam path. The filter changing apparatus can thus also introduce an empty filter receptacle into the optical passage and into the beam path. Likewise, instead of omitting an optical filter, free passage can also be made possible by a non-filtering optical element, such as a glass pane. A glass pane used as a window can also have an anti-reflective coating. The filter changing apparatus can in particular be integrated into a camera or can be connectable as a separate apparatus, e.g., designed as a snap-on filter, to the camera and/or an endoscope. For automatic filter change, the filter changing apparatus may have an operating element, such as a switch on its outer surface. Alternatively or in addition to the user visually detecting that the filter is being changed, the filter changing apparatus can also have a display element and/or a sensor-for example, a Hall-effect sensor.

An “optical filter” (also simply referred to as “filter”) is in particular an optical element which selects the incident radiation and/or incident beams on the basis of specific properties, such as a wavelength, a polarization state, an angle of incidence, and/or a direction of incidence, and thus allows them to pass through or prevents them from passing through. Likewise, an optical filter can change the properties of the light passing through it-for example, by converting circularly polarized light into linearly polarized light. In particular, an optical filter can block a specific spectral wavelength band. An optical filter may, for example, be a graduated filter, an edge filter, a polarizing filter, or an interference filter. An interference filter in particular has a coating which blocks light of a certain spectral range or allows it to pass through. The optical filter can in particular be used as an observation filter and/or detection filter, fluorescence observation filter, or excitation filter. The optical filter in particular comprises glass or a crystalline material. The optical filter may be planar or designed as a filter lens. In principle, instead of the optical filter, another optical element, such as a lens, an aperture, a polarizer, or a similar optical element, can also be arranged in the filter changing apparatus and/or the filter receptacle.

A “white light filter” is understood in particular to mean that a corresponding receptacle and/or position is devoid of an optical element, or an optical element in a corresponding receptacle and/or position is devoid of a filtering function, so that the light and/or white light is let through, in particular without being changed. White light is let through a white light filter in particular without changing its light properties, especially its wavelengths. A white light filter can also be a filter that blocks near-infrared light. A “white light filter” can also be a filter that filters the light to improve the quality of the image when illuminated with white light. For this purpose, a BG39 filter from Schott can be used, for example. Thus, by means of a white light filter, the light captured by an image sensor and/or a camera can also be adapted to a sensitivity curve and/or a specific sensitivity of the human eye.

A “fluorescence observation filter” (also referred to as a “fluorescence filter”) is in particular an optical, polychroitic interference filter for separating the emitted fluorescent light from the excitation light used. The fluorescence filter thus blocks the specific fluorescence excitation radiation and allows the fluorescence emission radiation to pass along the optical beam path. Preferably, the fluorescence filter completely blocks the excitation light while allowing the fluorescence emission light to pass through, which usually has a longer wavelength than the excitation light. Thus, a fluorescence filter is, in particular, an observation filter that filters out the excitation light that causes a fluorophore to glow. This is advantageous, because the excitation light is usually several orders of magnitude brighter than the resulting and/or emitted fluorescent light, and would otherwise outshine it. A fluorescence filter can also be a “blue filter,” “red filter,” “IR filter,” or “NIR filter.”

A “blue filter” is understood in particular to mean a filter which filters out the blue excitation light from a light source, but at least predominantly allows the fluorescent light, in particular fluorescent light emitted by a fluorophore, to pass through. For example, when using the fluorophore FITC (fluorescein isothiocyanate, green derivative of fluorescein), excitation occurs by means of LED at a wavelength of 460 nm, wherein longer-wavelength fluorescent light with a maximum at approximately 520 nm in the green spectral range is emitted by the fluorophore. In order to be able to clearly display the emitted fluorescent light of the FITC during imaging, the blue excitation light is filtered out using the filter changing apparatus and/or camera.

A “red filter” is understood in particular to mean a filter which filters out the red excitation light from a light source, but at least predominantly allows the fluorescent light, in particular fluorescent light emitted by a fluorophore, to pass through.

An “IR filter” is understood in particular to mean a filter which filters out infrared excitation light from a light source, but at least predominantly allows the fluorescent light, in particular fluorescent light emitted by a fluorophore, to pass through.

An “NIR filter” is understood in particular to mean a filter which filters out near-infrared excitation light from a light source, but at least predominantly allows the fluorescent light, in particular fluorescent light emitted by a fluorophore, to pass through.

A “filter center axis” is in particular the axis that passes through the center of the surface of the optical filter. In particular, the filter center axis is perpendicular to the surface of the optical filter. In the case of a circular optical filter, the filter center axis is arranged in particular in the center of the cross-section of the optical filter and is thus concentrically surrounded by the circular outer contour of the optical filter. The filter center axis is in particular substantially parallel to the axis of rotation and/or the optical axis.

An “optical passage” is in particular a hollow space in the filter changing apparatus through which light can pass. An optical passage is in particular a continuous opening through the housing, the housing parts, the rotatable drive wheel, and/or other components of the filter changing apparatus along the optical axis. The optical passage is in particular arranged around the center of the cross-section of the at least one housing part, the rotatable drive wheel, and/or around the optical axis. In particular, the optical passage is arranged concentrically around the optical axis. The optical passage extends in particular along the optical axis in the longitudinal direction. In particular, a filter receptacle and/or an optical filter can be arranged in front of and/or in the optical passage in the light propagation direction. Likewise, when light passes through, the optical passage may be free of an arranged optical filter and/or a receptacle. In principle, the optical passage can have any cross-sectional shape, but the optical passage is preferably circular in cross-section.

An “optical axis” is in particular a line along which a degree of rotational symmetry exists in an optical system. The optical axis is in particular an imaginary line that defines a path along which light propagates through the filter changing apparatus and/or the camera towards an image sensor. Preferably, the optical axis runs through the center of curvature of each pivoted-in filter and/or of a downstream lens system and/or objective lens system. However, the optical axis can also be bent and/or directed by a lens, an optical element, and/or one of the optical filters. The optical beam path as a geometric course of light beams is in particular arranged in and/or around the optical axis and runs along, converging and/or dispersing with respect to, the optical axis.

In principle, it should be pointed out that the terms “first” and “second” filter holder arm, filter, and other terms are used only for differentiation. For example, when the drive gear is rotated, the filter receptacle of the associated filter holder arm that is the first to be pivoted into the optical passage depends upon whether the drive gear is rotated clockwise or counterclockwise.

A “filter holder arm” (also simply called a “filter holder”) is in particular an elongated element and/or an arm which is rotatably arranged around its axis of rotation on and/or on an inner side of a housing part. In principle, the filter holder arm can have any shape. Preferably, the filter receptacle is arranged at the widest end of the filter holder arm, wherein, in this portion, the outer diameter of the filter holder arm is in particular only slightly larger than the diameter of the received optical filter. Proceeding from the filter receptacle, the filter holder arm can taper gradually and/or continuously towards the axis of rotation. For example, the filter holder arm can be drop-shaped or club-shaped. The filter holder arm can be formed in one plane or portions thereof can be formed in two or more planes. The filter holder arm can also have different material thicknesses. The filter holder arm in particular has a bore on its underside, in which bore the rotary shaft is arranged. The rotary shaft can in particular be integrally bonded and/or non-positively connected in this bore-for example, by being pressed in. As a result of a material thickness of the filter holder transverse to its longitudinal direction, this bore may be partially or completely continuous. The other end of the rotary shaft, opposite the bore, is in particular connected and/or fastened to one of the housing parts. The rotary shaft and thus the axis of rotation of each filter holder arm is arranged in particular at the end and/or on the opposite side to the filter receptacle of the filter holder arm in the longitudinal direction of the filter holder arm. In principle, the two or more filter holder arms can have the same and/or different lengths. The longitudinal direction of each filter holder arm is in particular substantially transverse to the optical axis. In particular, a filter gear is connected to the rotary shaft of each filter holder arm. Preferably, the filter gear surrounds the rotary shaft. Each filter gear is connected to the respective rotary shaft in particular so as to transmit movement, such that a rotational movement of the filter gear is directly converted via the connected rotary shaft into a rotation of the entire filter holder arm. The rotary shafts of two or more filter holder arms are in particular parallel to one another. Preferably, the rotary shafts and thus the axes of rotation are arranged at equal distances from the optical passage on the first housing part. The rotary shafts and/or axes of rotation of two or more filter holder arms can be arranged on one of the housing parts, in particular distributed in the shape of a circle when viewed in cross-section. The rotary shafts and/or axes of rotation are arranged in particular in relation to the teeth of the drive wheel such that the teeth of the filter gears each engage directly or indirectly with the teeth of the rotary drive gear. Above all, the rotary shafts and/or axes of rotation of the filter holder arms are arranged in such a way that the teeth of each filter gear cannot mesh with one another, but engage only with the teeth of the drive gear. Preferably, the filter holder arms are coordinated with one another in terms of their shape, e.g., by means of rounded and/or narrower portions, and their arrangement such that the filter holder arms do not touch one another during rotation, and as little play as possible is provided. Preferably, each filter holder is designed to be stepped in two flat portions with different heights and/or material thicknesses. This also allows the filter holders to move at least partially over one another and thus overlap without touching. The shape and/or dimensions of each filter holder arm depend in particular upon the diameter of the optical filter and the optical design.

A “filter gear” is in particular a gear which has teeth distributed all the way around its outer circumference. The filter gear has in particular a continuous bore in the center, through which the rotary shaft is guided. Likewise, the rotary shaft of the filter holder arm can, however, also only connect the filter holder arm itself to the filter gear and not be designed to completely pass through the filter gear to the housing part. In this case, the filter gear has a lower bore in which, for example, a cylindrical pin fastened to the housing part is arranged, such that the filter gear can be rotated around the cylindrical pin and thus the entire filter holder arm.

An “axis of rotation” is in particular an axis to which the filter holder arm is connected and around which the filter holder arm rotates. The axis of rotation is in particular the straight line which remains stationary during a rotational movement of a filter holder arm. The axis of rotation is in particular a central longitudinal axis of the rotary shaft. In particular, the axis of rotation is parallel to the optical axis.

A “filter receptacle” (also called a “receptacle”) is in particular a hollow body or hollow space with a partial or complete external enclosure and/or border, into which an optical filter can be inserted and which at least partially encloses the optical filter around its circumference and holds it. A filter receptacle may, for example, be a short tubular body. The filter receptacle forms in particular a holder and/or a protective casing for the optical filter. A filter receptacle can, for example, also have a spring washer to hold the optical filter.

A “drive gear” is in particular a disc or a ring which has at least one circumferential toothing. The drive gear is in particular a planar, flat component, the opposing surfaces of which are substantially perpendicular to the optical axis and/or the optical passage. The drive gear has in particular an external toothing and/or an internal toothing. The drive gear can in particular also be designed as a drive wheel and can thus be externally driven and rotated, in particular by hand, by means of a drive unit, and/or a motor. For this purpose, the outer circumferential surface of the drive gear may, for example, be designed as a contact surface and driven by a drive unit and/or a transmission acting at and/or upon this contact surface. As a contact surface, the drive gear can, for example, have an external toothing in which a drive engages from the outside. In addition to an optional toothing for driving the drive gear itself, the drive gear in this embodiment has in particular an internal toothing the entire way around for transmitting its rotational movement to the filter gears. An internal toothing on the inside of the drive wheel is in particular arranged concentrically around the optical passage. Thus, a space between the optical passage and the internal toothing of the drive wheel in particular forms a space for receiving the filter gears of the filter holder arms. Depending upon whether the internal or external toothing of the drive gear engages directly or indirectly with the filter gears, the rotary shafts and/or cylindrical pins of each of the filter holder arms are arranged at a defined distance from the internal or external toothing of the drive gear. If the drive gear is designed having an external toothing for driving the filter gears, the drive gear is arranged in particular around the optical passage. In this case, the drive gear can in particular be fastened to a filter wheel, the outer circumferential surface of which acts in particular as a driving means. According to one design of the drive gear having an external toothing for engaging with the filter gears of the filter holder arms, the rotary shafts of the filter holder arms are arranged around the outside of the external toothing of the drive gear at a distance therefrom. Consequently, in this case, the filter holder arms are further away from the optical passage. In contrast, the embodiment of the drive wheel having an internal toothing for engaging with the filter gears has the advantage that the filter holder arms are closer to the optical passage and can therefore be pivoted into the optical passage more quickly. In addition, if the drive gear is designed having a driving internal toothing, the entire drive gear can be designed as a drive and/or filter wheel and can be driven directly on its outer circumference. Consequently, the embodiment of the drive gear having an internal toothing for driving the filter gears has a smaller size. The drive gear can in particular be rotatably held, and is thereby mounted, between the two housing parts. In particular, the drive gear is infinite and can therefore rotate clockwise and/or counterclockwise any number of times. Accordingly, the filter holder arms can also be infinitely rotated in either of the two directions of rotation as a result of the transmission via each filter gear and the rotary shaft. In order to minimize the frictional forces when the teeth engage, the filter gears and the drive gear can in particular have a material with a low coefficient of friction-for example, aluminum and/or a polymeric material such as PTFE.

A “housing part” is in particular a component of the filter changing apparatus on and/or to which the at least two filter holder arms are rotatably fastened. The housing part can be a distal or proximal housing part and/or a distal or proximal housing cover of the filter changing apparatus. The housing part can also be a base plate inside the housing. Likewise, a base plate can be arranged for the intermediate storage and passage of the rotary shafts. The two housing parts are connected to one another in particular in such a way that the drive gear and/or drive wheel arranged therebetween can rotate freely. For this purpose, for example, at least one connecting piece spaced apart from the circumferential surface of the drive gear can connect the two housing parts in a direction that is parallel to the optical axis, wherein the free end of the connecting piece can be fastened to the other housing part-for example, by means of a screw connection. Likewise, the two housing parts can be connected to one another inside the housing. This means that the drive gear and/or drive wheel can also have a larger diameter than one or both of the housing parts.

A “camera” (also called a “camera head”) is in particular a piece of equipment for receiving image light along an optical axis from an endoscope and for focusing the received image light on at least one image sensor. In addition to the at least one image sensor, the camera may in particular have an aperture or a window for letting through the received image light and a lens system for focusing the image light on the at least one image sensor. The image data recorded by at least one image sensor can in particular be transmitted electronically by the camera head to a display system and/or to an image processing unit in order to display the endoscopic image to the user. The camera may have means for recognizing the connected endoscope and for processing algorithms. A connector for connecting an endoscope to the camera can be arranged at the distal end of the endoscope and/or the proximal end of the camera head. The filter changing apparatus according to the invention itself may also be designed as a connector for connecting an endoscope to a camera.

An “endoscope” is in particular a medical or industrial piece of equipment for endoscopic examination and inspection of a human or animal body cavity and/or an industrial cavity, such as a tube. The endoscope in particular has a handpiece, a shaft, a light source, a light guide, a sensor, and/or a camera. The endoscope is in particular a video endoscope, which has digital image recording and image transmission and thus an integrated or connectable camera. In addition to medical and veterinary applications, an endoscope and/or video endoscope may, however, also be used for industrial purposes-for example, for visual inspection in hard-to-reach cavities. In industrial applications, an endoscope is often referred to as a borescope.

An “image sensor” is in particular a light-sensitive electronic component which is based upon an internal photoelectric effect. By means of the image sensor, one or more images from the viewing area of the imaging apparatus are in particular recorded and converted into electronic signals. The image sensor has a sensor plane in the image plane of the optical system, of a lens system, and/or of an objective lens. An electronic image sensor may in particular be a CCD sensor (charge-coupled device) or a CMOS sensor (complementary metal-oxide semiconductor).

“Distal-side” and “distal” mean in particular an arrangement and/or a corresponding end or portion that is remote from the user. Accordingly, when the filter changing apparatus is connected to an endoscope, the endoscope is arranged on the distal side. Accordingly, “proximal-side” or “proximal” means an arrangement or a corresponding end or portion near the user. When the filter changing apparatus is connected to a camera and/or a camera head, the camera and/or the camera head is/are arranged on the proximal side of the filter changing apparatus.

In a further embodiment, the filter changing apparatus has a third filter holder arm, a fourth filter holder arm, a fifth filter holder arm, and/or optionally more filter holder arms.

This allows additional, different filters or a combination of the same and different filters to be pivoted into and out of the optical beam path when the drive gear is rotated. Depending upon the desired frequency of use, a frequently used filter can, for example, be pivoted into and out of the optical passage several times during one revolution of the drive filter gear. When using five filter arms, for example, three fluorescence filters, one white light filter, and one empty filter slot for multispectral imaging can each be arranged in a filter receptacle of the five filter holder arms. Likewise, the same or different fluorescent filters can, however, also alternate with a subsequent white light filter.

Since the filter changing apparatus is driven by only a single drive gear and the filter gears are moved thereby, the filter changing apparatus can be easily scaled and implemented for a different number of filter holder arms, due to the inner or outer diameter of the drive gear and thus the length of the circumferential toothing. Consequently, the filter changing apparatus and its drive can be easily and reliably automated, largely independently of the number of filter holder arms.

In order to ensure a compact size of the filter changing apparatus and collision-free movements of the filter holder arms, the distance between the filter center axis and the axis of rotation of a filter holder arm can be 0.80-times to 1.10-times the maximum outer diameter of the filter holder arm in the region of the filter receptacle.

In a further embodiment of the filter changing apparatus, the distance between two rotation axes of two filter holder arms is 1.05-times to 1.50-times the maximum outer diameter of the respective filter holder arm in the region of the filter receptacle.

Thus, the distance between the rotation axes of two adjacent filter holder arms is only slightly larger than the maximum outer diameter of each filter holder arm. Consequently, the available space provided by the drive gear and the optical passage can be optimally utilized for arranging and/or moving the filter holder arms.

In order for a filter holder arm to be at least partially slidable and/or movable over and/or under an adjacent other filter holder arm, each filter holder arm can have a first arm portion having the rotary shaft arranged thereon and a second arm portion having the filter receptacle arranged thereon, wherein the two arm portions are connected by means of a transition portion.

Thus, the two arm portions of each filter holder arm can be arranged at different heights of the filter holder arm in a direction along the axis of rotation, and thus at different distances from one of the housing parts.

The “first arm portion” and the “second arm portion” are designed differently, in particular in the longitudinal direction of the filter holder arm and/or along the axis of rotation. In particular, the two arm portions can have different shapes, rounded portions, and/or material thicknesses. For example, the first arm portion having the rotary shaft arranged thereon can be substantially oval in cross-section transverse to the axis of rotation, and the second arm portion having the filter receptacle can be circular. Preferably, the second arm portion having the filter receptacle has a larger diameter than the first arm portion. A different height is also understood to mean that, when rotary shafts are arranged on the distal housing part, a proximal surface of one arm portion is arranged further in the proximal direction than a proximal surface of the other arm portion. Likewise, when rotary shafts are arranged on the proximal housing part, a distal surface of one arm portion can be arranged further in the distal direction than the distal surface of the other arm portion.

A “transition portion” is in particular a portion between the first arm portion and the second arm portion, which connects the two arm portions and/or aligns the different properties of the two arm portions in the transition. The transition portion may in particular be a shaped and/or curved portion of the filter holder arm, which, for example, connects the different heights of the first arm portion and the second arm portion. In principle, it should be pointed out that the filter holder arm and its portions can be formed in several parts, connected, or integrally formed. For example, the filter holder arm can be cast as one component. Likewise, the two arm portions and the transition portion can, for example, also each be integrally bonded—for example, by gluing.

In a further embodiment of the filter changing apparatus, the first arm portion and the second arm portion are spaced apart from one another in a direction along the axis of rotation.

Thus, the first arm portion and the second arm portion are stepped relative to one another and/or spaced apart from the housing part at different heights.

In principle, it should be emphasized that the first arm portion can be arranged closer to the housing part along the axis of rotation than the second arm portion, or vice versa. However, due to the axis of rotation, the first arm portion is preferably arranged closer to the housing part, and thus the second arm portion is higher and thus further away from the housing part.

In order for two adjacent filter arms to be able to move at least partially on overlapping circular radii, the second arm portion can be arranged at a greater height than the height of the first arm portion in a direction along the axis of rotation such that a second arm portion of a first filter holder arm can be arranged and/or rotated at least partially over a first arm portion of a second filter holder arm.

As a result, a plurality of filter holder arms can be arranged more compactly in the available installation space, e.g., between the optical passage and the drive gear, and/or the number of filter holder arms can be easily increased in an existing filter changing apparatus without further modifications to the filter changing apparatus.

In a further embodiment of the filter changing apparatus, the difference between the maximum height of the second arm portion and the maximum height of the first arm portion is 1.00-times to 1.50-times the maximum material thickness of the second arm portion in a direction along the axis of rotation.

In addition to possible manufacturing tolerances, there is thus sufficient free space along the axis of rotation between an underside of the second arm portion having the filter receptacle of a first filter holder arm and the upper side of the first arm portion of a second filter holder arm when the axis of rotation is arranged vertically.

In order to axially secure each rotary shaft, each rotary shaft can be connected to the at least one housing part by means of a securing element.

A “securing element” can, for example, be an adjusting ring or an axial securing ring.

In a further embodiment of the filter changing apparatus, the drive gear has an internal toothing and/or an external toothing.

If the drive gear has both an internal toothing and an external toothing, the external toothing can be used to drive the drive gear by means of a transmission and/or motor, and the internal toothing can be used to drive the filter gears. In principle, the drive gear can also be connected and/or combined with a drive wheel. For this purpose, for example, the drive gear can be arranged around the optical passage of the filter changing apparatus with an external toothing for driving the filter gears on a distal or proximal surface of a drive wheel, and the drive wheel itself can have an external toothing on its outer diameter for driving by means of a transmission and/or motor.

In order to mount the drive gear in a simple manner and to make it rotatable, the filter changing apparatus can have a second housing part, and the first housing part and the second housing part can be connected to one another in their interior and/or on their exterior so that the drive gear can be freely externally driven and/or rotated across its entire circumference.

This allows the drive gear to rotate freely and its outer circumference to be freely contactable.

In a further embodiment, the filter changing apparatus has a motor for driving the rotatable drive gear.

In a further aspect of the invention, the object is achieved by a camera head for an endoscope, wherein the camera head has an image sensor, an opening for receiving light of an image along an optical path, and an optical lens system for focusing the light on the image sensor, wherein the camera head has at least one filter changing apparatus as described above.

Thus, a camera head is provided on or in which a compact, space-saving filter changing apparatus is arranged, which enables quick filter change and different filters to be rotated through one after the other. If at least one filter changing apparatus is arranged directly in the camera head, the camera head can be detachably connected to different types of endoscopes. For example, the filter changing apparatus can be integrated into the camera head by installing it instead of a bayonet lock. This can minimize the extent to which the beam path is lengthened. Of course, the camera head can also have two or more filter changing apparatuses in series in an optical path and/or along the optical axis, if the simultaneous use of two or more filters in the beam path is desired, in particular in multispectral imaging and/or in a broad application of different fluorophores in fluorescence imaging.

In order to adjust a currently used filter configuration or adapt it to desired different observation modes, the camera head may comprise a detection unit for detecting an identification of each optical filter in the optical path. Likewise, the camera head can have a control unit for adjusting the rotational speed of the drive gear and for checking and/or adjusting each optical filter arranged in the beam path according to the operating mode selected at the time.

In a further aspect of the invention, the object is achieved by a retrofit kit for retrofitting a camera head and/or an endoscope, wherein the retrofit kit has at least one filter changing apparatus as described above, such that the filter changing apparatus can be arranged between a proximal end of the endoscope and a distal end of the camera head.

This provides a retrofit kit (also referred to as an “adapter”) with at least one filter changing apparatus, which retrofit kit simultaneously serves both as a connector between an existing endoscope and an existing camera head and to make different observation modes possible. In addition, the retrofit kit may also comprise two or more filter changing apparatuses arranged in series between the endoscope and the camera head, or one filter changing apparatus may be replaced by another filter changing apparatus to make different applications and/or observation options possible.

### BRIEF DESCRIPTION OF THE DRAWINGS

The invention will be explained below on the basis of exemplary embodiments. In the figures:

FIG. 1 is a schematic three-dimensional detail view of an endoscope system with an endoscope, a filter changer, and a camera head,

FIG. 2 is a three-dimensional representation of a filter changer in side view with an endoscope receptacle and a camera receptacle,

FIG. 3 is a schematic view of a distal cover half of the filter changer from FIG. 2,

FIG. 4 is a schematic view of a proximal cover half of the filter changer,

FIG. 5 is a three-dimensional view of the filter changer in the open state and in which five filter holder arms and a drive gear can be seen,

FIG. 6 is a three-dimensional side view of the filter changer in the open state from FIG. 5,

FIG. 7 is a three-dimensional side view of a filter holder arm,

FIG. 8 is a plan view of the filter holder arm from FIG. 7, and

FIG. 9 is a three-dimensional cross-sectional view of the filter changer.

### DETAILED DESCRIPTION

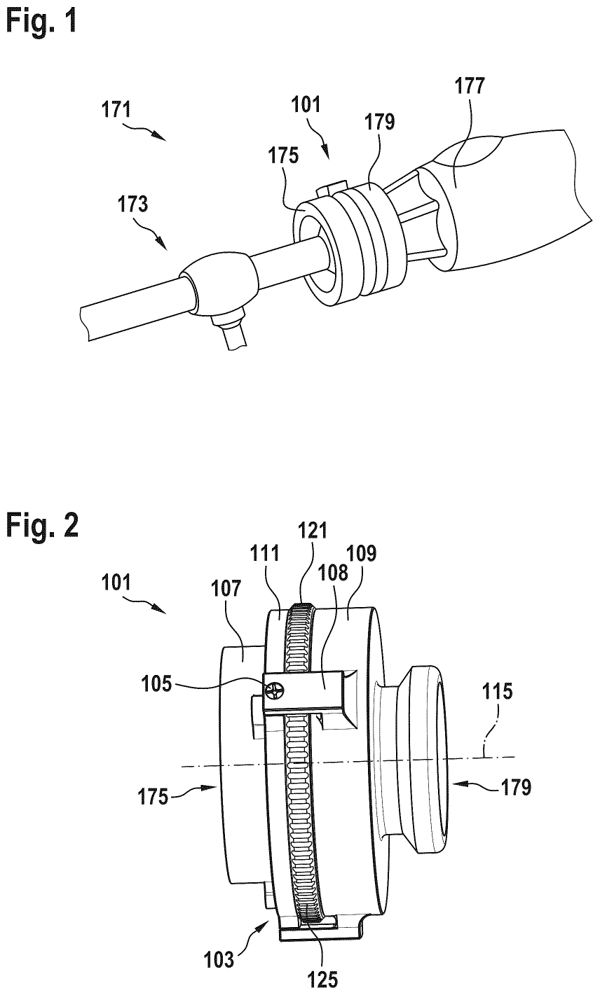

An endoscope system 171 has a camera head 177, a filter changer 101, and an endoscope 173. The filter changer 101 is connected to the camera head 177 by means of a camera receptacle 179 and to the endoscope 173 by means of an endoscope receptacle 175 (FIGS. 1 and 2).

The filter changer 101 has a housing 103 having a distal cover half 107 with a base plate 111 and a proximal cover half 109, wherein a drive gear 121 is rotatably arranged between the base plate 111 and the proximal cover half 109. The proximal cover half 109 has on its outer circumference three evenly distributed connecting pieces 108, which are guided in their longitudinal direction along an optical axis 115 to the outer circumference of the base plate 111 at a distance from an outer diameter of the drive gear 121, where they are each fastened by means of inwardly directed screws 105. As a result, the drive gear 121 is rotatably mounted between the base plate 111 and the proximal cover half 109 and can be driven around its entire circumference by its external toothing 125 (FIG. 2).

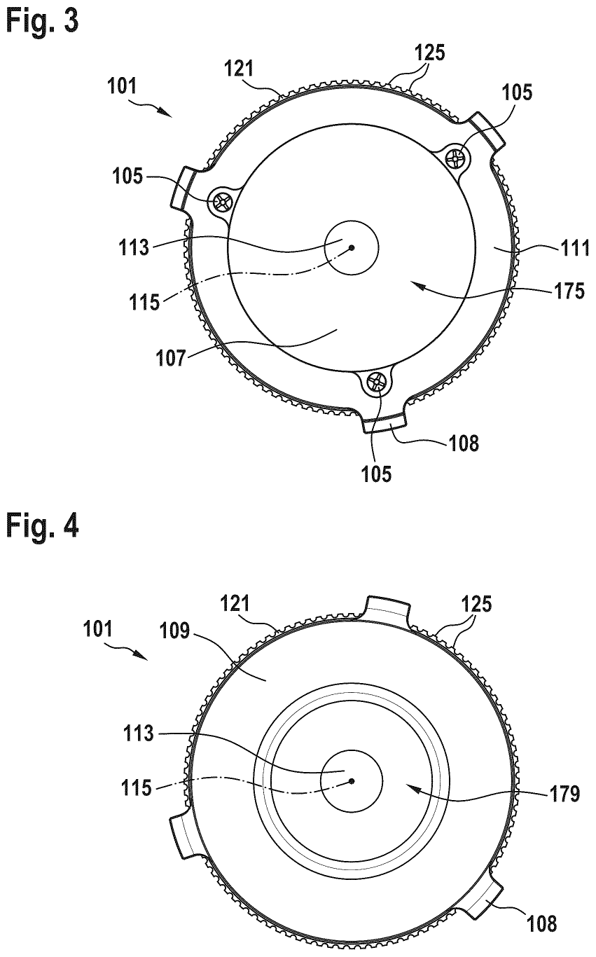

The distal cover half 107 has the endoscope receptacle 175 on the distal side, and the proximal cover half 109 has the camera receptacle 179 on the proximal side (FIG. 3). The distal cover half 107 and the proximal cover half 109 each have an optical passage 113 that is concentric with the optical axis 115. Likewise, the base plate 111 has an optical passage 113.

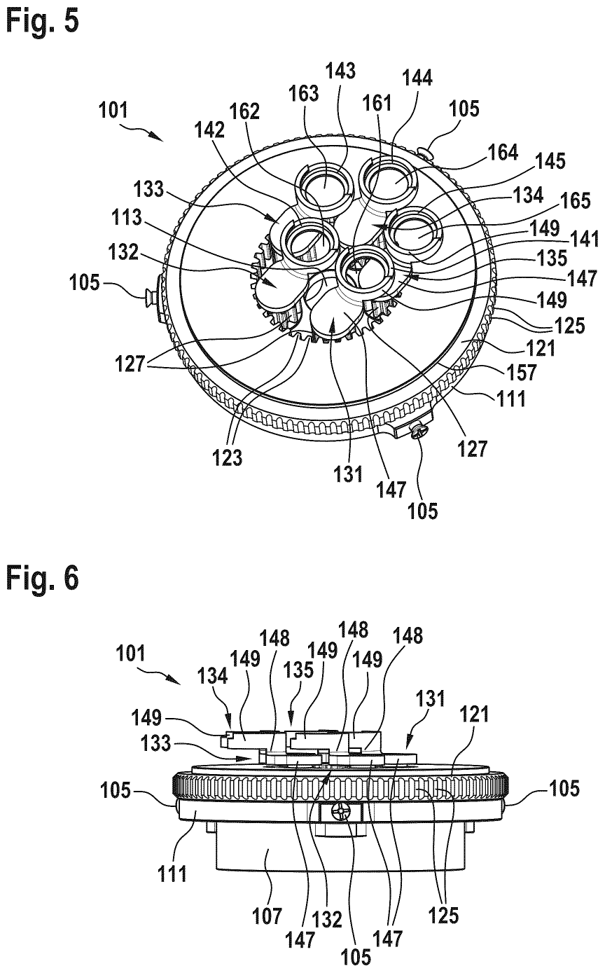

FIG. 5 shows the filter changer 105 with the proximal cover half 109 removed. A first filter holder arm 131 having a first filter receptacle 141 and a received first filter 161, a second filter holder arm 132 having a second filter receptacle 142 and a received second filter 162, a third filter holder arm 133 having a third filter receptacle 143 and a received third filter 163, a fourth filter holder arm 134 having a fourth filter receptacle 144 and a received fourth filter 164, and a fifth filter holder arm 135 having a fifth filter receptacle 145 and a received fifth filter 165 are arranged in a space between an internal toothing 123 of the drive gear 121 and the optical passage 113. The filter holder arms 131 to 135 are rotatable and movable within a movement circle 175.

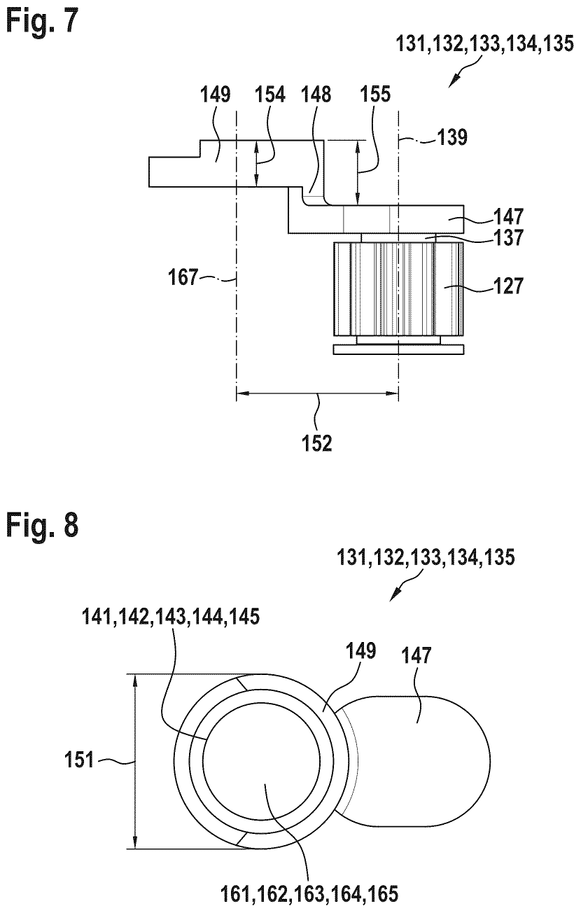

Each filter holder arm 131 to 135 is stepped, having a first arm portion 147 followed by a transition portion 148 and a second arm portion 149 (FIGS. 7 and 8). The first arm portion 147 has an oval cross-section and is connected to a rotary shaft 137 on its underside. A filter gear 127 is fastened around the rotary shaft 137. The rotary shaft 137 has an axis of rotation 139 which is perpendicular to the longitudinal orientation of each filter holder arm 131 to 135. The second arm portion 149 is circular when viewed from above, wherein a filter 161, 162, 163, 164, 165 is accommodated in each of the filter receptacles 141 to 145.

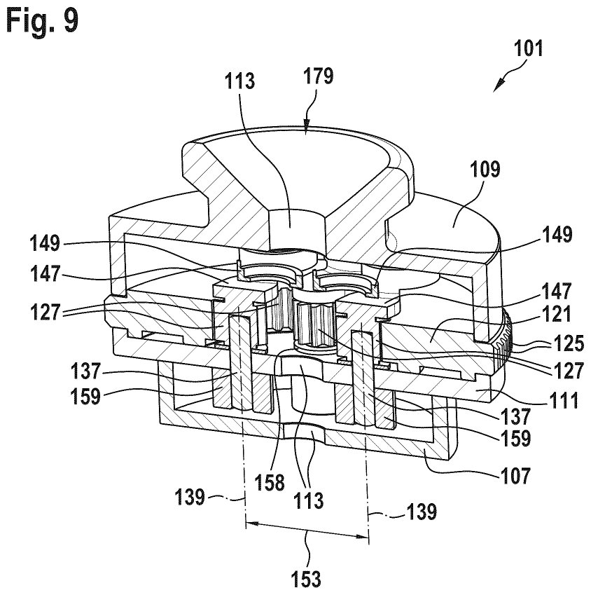

A maximum outer diameter 151 of the second arm portion 149 is 9.4 mm and is larger than a maximum outer diameter of the first arm portion 147. The maximum material thickness 154 of the second arm portion 149 is 2.5 mm, and a step height 155 between an upper side of the second arm portion 149 and an upper side of the first arm portion 147 is 3.5 mm, so that there is a distance of 1.0 mm between the underside of a second arm portion 149 of a filter holder arm and the upper side of a first arm portion 147 of a second filter holder arm arranged next to it. A distance 152 between a filter center axis 167 of each received filter 161 to 165 and the respective axis of rotation 139 is 8.8 mm. Each axis of rotation 139 is guided through a washer 158 and the base plate 111 and is axially secured on the inner side on the inside of the distal cover half 107 by means of an adjusting ring 159 (see FIG. 9). A distance 153 between two adjacent axes of rotation 139 is 10.35 mm in each case. Each of the axes of rotation 139 of all five filter holders 131 to 135 are arranged at a distance from the internal toothing 123 of the drive gear 121 such that the teeth of each filter gear 127 engage directly with the internal toothing 123 of the drive gear 121.

The following operations are carried out by means of the filter changer 101 and the endoscope system 171.

By means of a motor and gear wheel (not shown), the drive gear 121 is driven by its external toothing 125 and made to rotate clockwise. Due to the engagement between the internal toothing 123 of the drive wheel 121 and the filter gear 127 of each of the five filter holder arms 131, 132, 133, 134, 135, these filter holder arms are made to move in a circular motion, due to the transmission of the rotational movement of each filter gear 127 via the rotary shaft 137, and move along the internal toothing 123 in a clockwise direction, wherein the second arm portion 149 also performs a corresponding circular movement in a clockwise direction each time.

In the state shown in FIG. 5, the first filter holder arm 131 is being pivoted out of the optical passage 113, and, due to the step height 155, the second arm portion 149 of the first filter holder arm 131 pivots with the first filter receptacle 141 and the first filter 161 over the first arm portion 147 of the fifth filter holder 135 without touching it. Upon further rotation of the drive wheel 121 in the clockwise direction, the second filter holder arm 132 is pivoted into the optical passage 113, and the first filter holder arm 131 simultaneously moves further outwards towards the movement circle 157. The third filter holder arm 133, the fourth filter holder arm 134, and the fifth filter holder arm 135 analogously continue to move accordingly.

Thus, a filter changer 101 is provided with which, due to the filter holder arms 131 to 135 being spatially arranged near to one another and moving at the same time on the inside of the internal toothing 123, different filters 161 to 165 can be quickly pivoted one after the other into the optical passage 113, wherein the direction of rotation of the drive wheel 121 can be changed by the user at any time. Depending upon which filter 161 to 165 is selected, e.g., three fluorescence filters, a white light filter, and optionally also an empty filter receptacle for multispectral observation, the user can quickly and easily implement various observation modes and use the filter changer 101 with different types of endoscopes 173 and camera heads 177.

### LIST OF REFERENCE SIGNS

101 filter changer
103 housing
105 screw
107 distal cover half
108 connecting piece
109 proximal cover half
111 base plate
113 optical passage
115 optical axis
121 drive gear
123 internal toothing
125 external toothing
127 filter gear
131 first filter holder arm
132 second filter holder arm
133 third filter holder arm
134 fourth filter holder arm
135 fifth filter holder arm
137 rotary shaft
139 axis of rotation
141 first filter receptacle
142 second filter receptacle
143 third filter receptacle
144 fourth filter receptacle
145 fifth filter receptacle
147 first arm portion
148 transition portion
149 second arm portion
151 maximum outer diameter of the second arm portion
152 distance between filter center axis and axis of rotation
153 distance between two axes of rotation
154 maximum material thickness of the second arm portion
155 step height
157 movement circle
158 washer
159 adjusting ring
161 first filter
162 second filter
163 third filter
164 fourth filter
165 fifth filter
167 filter center axis
171 endoscope system
173 endoscope
175 endoscope receptacle
177 camera head
179 camera receptacle

1. A filter changing apparatus for an endoscopic camera, wherein the filter changing apparatus bescomprises:
a housing having at least one first housing part
an optical passage along an optical axis,
a rotatable drive gear, and
at least two filter holder arms each having a filter receptacle for an optical filterhaving a filter center axis, wherein the at least two filter holder arms are each rotatably arranged on the first housing part by means of a rotary shaft having an axis of rotation, wherein the respective axis of rotation is spaced apart from the filter center axis of each filter holder arm and the axes of rotation are arranged in parallel with one another, wherein on each filter holder arm, a filter gear is arranged around the respective rotary shaft, and the rotatable drive gear can engage in each filter gear, so that, when the rotatable drive gear is driven solely by engaging with the filter gears, the at least two filter holder arms can be simultaneously rotated and/or pivoted into and/or out of the optical passage.

2. The filter changing apparatus according to claim 1, wherein the filter changing apparatus has a third filter holder arm, a fourth filter holder arm, a fifth filter holder arm, and/or optionally further filter holder arms.

3. The filter changing apparatus according to claim 1, wherein the distance between the filter center axis and the axis of rotation of a filter holder arm is 0.80-times to 1.10-times the maximum outer diameter of the filter holder arm in the region of the filter receptacle.

4. The filter changing apparatus according to claim 1, wherein the distance between two axes of rotation of two filter holder arms is 1.05-times to 1.50-times the maximum outer diameter of the respective filter holder arm in the region of the filter receptacle.

5. The filter changing apparatus according to claim 1 wherein each filter holder arm has a first arm portion having the rotary shaft arranged thereon and a second arm portion having the filter receptacle arranged thereon, wherein the two arm portions are connected by means of a transition portion.

6. The filter changing apparatus according to claim 5, wherein the first arm portion and the second arm portion-are spaced apart from one another in a direction along the axis of rotation.

7. The filter changing apparatus according to claim 5, wherein the second arm portion is arranged at a greater height than the height of the first arm portion in a direction along the axis of rotation-so that a second arm portion of a first filter holder arm can be arranged and/or rotated at least partially over a first arm portion of a second filter holder arm.

8. The filter changing apparatus according to claim 1, wherein the difference between the maximum height of the second arm portion and the maximum height of the first arm portion is 1.00-times to 1.50-times the maximum material thickness of the second arm portion in a direction along the axis of rotation.

9. The filter changing apparatus according to claim 1, wherein each rotary shaft is connected to the at least one housing part by means of a securing element.

10. The filter changing apparatus according to claim 1, wherein the drive gear has an internal toothing and/or an external toothing.

11. The filter changing apparatus according to claim 1, wherein the filter changing apparatus has a second housing part, and the first housing part and the second housing part are connected to one another in their interior or on their exterior so that the drive gear can be freely externally driven and/or rotated over its entire circumference.

12. The filter changing apparatus according to claim 1, wherein the filter changing apparatus has a motor for driving the rotatable drive gear.

13. A camera head for an endoscope, wherein the camera head has an image sensor, an opening for receiving light of an image along an optical path, and an optical lens system for focusing the light on the image sensor, wherein the camera head has at least one filter changing apparatus according to claim 1.

14. A retrofit kit for retrofitting a camera head and/or an endoscope, wherein the retrofit kit has at least one filter changing apparatus according to claim 1, such that the filter changing apparatus can be arranged between a proximal end of the endoscope and a distal end of the camera head.
